(19) United States
(12) Patent Application Publication
(10) Pub. No.: US 2026/0345558 A1
(43) Pub. Date: Nov. 5, 2026

(54) System and Method for Inhibiting the Nain Protease (Mpro) of SARS-CoV-2

(71) Applicant: Dawn Solomon, Missouri, TX (US)

(72) Inventor: Dawn Solomon, Missouri, TX (US)

(21) Appl. No.: 19/196,323

(22) Filed: May 1, 2025

(51) Int. Cl. A61K 31/4184

(52) U.S. Cl. CPC A61K 31/4184

(57) ABSTRACT

A novel benzimidazole-based compound designed to inhibit the main protease (Mpro) of SARS-CoV-2, the virus responsible for COVID-19 a method of manufacture, and method of use is disclosed. The compound features a benzimidazole core with a fluorine atom at the 7-position, facilitating pi-stacking interactions with His41 in the Mpro active site. The chiral side chain includes a modified amphetamine backbone with a chiral carbon bearing a methyl group and an isobutyl group, filling the hydrophobic S2 pocket of Mpro. The nitrile warhead forms a covalent bond with the catalytic Cys145 of Mpro, ensuring irreversible inhibition. Additionally, the glutamine-mimicking side chain forms hydrogen bonds with Gln189, Glu166, and Thr190 in the Mpro active site. The compound can be administered in a pharmaceutical composition to treat COVID-19.

### BACKGROUND

This disclosure relates to a system and method for inhibiting the main protease (Mpro) of SARS-CoV-2. For purposes of this disclosure, specific examples of the systems and methods are discussed. However, such discussions are solely exemplary and not limiting.

The COVID-19 pandemic, caused by the SARS-CoV-2 virus, has led to unprecedented global health challenges. SARS-CoV-2 is a highly contagious virus that primarily affects the respiratory system, leading to severe illness and, in many cases, death. The virus's ability to rapidly mutate and spread has made it difficult to control, necessitating the development of effective treatments and preventive measures.

One of the critical components in the viral replication cycle of SARS-CoV-2 is the main protease (Mpro), also known as 3CLpro. Mpro plays a vital role in processing the polyproteins that are translated from the viral RNA, which is essential for the virus's replication and transcription. Inhibiting Mpro can effectively halt the replication of the virus, making it a prime target for antiviral drug development.

Despite the availability of some antiviral treatments, there remains a significant need for new inhibitors that can target Mpro with improved potency and broad-spectrum activity against various coronaviruses, including SARS-CoV-1 and MERS-CoV. Additionally, these inhibitors should be safe for use, with minimal impact on physiological parameters such as body pH levels.

It is for these reasons that a system for inhibiting the main protease (Mpro) of SARS-CoV-2 would be useful.

### SUMMARY

The disclosure teaches a system for inhibiting the main protease (Mpro) of SARS-CoV-2, comprising a benzimidazole-based compound derived from an amphetamine scaffold. The benzimidazole core includes a fluorine atom at the 7-position, facilitating pi-stacking interactions with His41 in the Mpro active site. The chiral side chain includes a modified amphetamine backbone with a chiral carbon bearing a methyl group and an isobutyl group, which fills the hydrophobic S2 pocket of Mpro. The nitrile warhead forms a covalent bond with the catalytic Cys145 of Mpro, ensuring irreversible inhibition. Additionally, the glutamine-mimicking side chain forms hydrogen bonds with Gln189, Glu166, and Thr190 in the Mpro active site. The compound is administered in a pharmaceutical composition to treat COVID-19 and related coronavirus infections, with the pharmaceutical composition being an oral tablet with a dose of 10-100 mg. Furthermore, the compound may be modified with a histidine-mimicking side chain to target picornavirus 3Cpro.

The disclosure also teaches a method for synthesizing a benzimidazole-based compound for inhibiting the main protease (Mpro) of SARS-CoV-2. The synthesis involves starting with 2-nitro-4-fluoroaniline and reducing the nitro group to an amine. Cyclization with formic acid yields 7-fluorobenzimidazole. A Suzuki coupling at C5 using (R)-2-methyl-3-bromopropionitrile is performed. The isobutyl group is added to the chiral carbon using isobutylmagnesium bromide. The synthesis of the glutamine-mimicking side chain starts with serine, protecting the hydroxyl, converting the carboxyl to amide, and deprotecting the hydroxyl. Lithiation of C2 of benzimidazole with n-BuLi and quenching with CO2 forms a carboxyl group. Coupling with H2NCH2CH(OH)CONH2 using EDC/HOBt completes the synthesis. The final product is 7-fluorobenzimidazole(5)-CH(CH3)(CH2CH(CH3)2)-CH2-CN-CONHCH2CH(OH)CONH2.

The disclosure also includes a method for inhibiting the main protease (Mpro) of SARS-CoV-2, comprising administering a pharmaceutical composition containing a benzimidazole-based compound derived from an amphetamine scaffold to a subject in need thereof. The benzimidazole core includes a fluorine atom at the 7-position, facilitating pi-stacking interactions with His41 in the Mpro active site. The chiral side chain includes a modified amphetamine backbone with a chiral carbon bearing a methyl group and an isobutyl group, filling the hydrophobic S2 pocket of Mpro. The nitrile warhead forms a covalent bond with the catalytic Cys145 of Mpro, ensuring irreversible inhibition. Additionally, the glutamine-mimicking side chain forms hydrogen bonds with Gln189, Glu166, and Thr190 in the Mpro active site. The compound is administered in a pharmaceutical composition to treat COVID-19 and related coronavirus infections, with the pharmaceutical composition being an oral tablet with a dose of 10-100 mg. Furthermore, the compound may be modified with a histidine-mimicking side chain to target picornavirus 3Cpro 1.

### BRIEF DESCRIPTION OF THE DRAWINGS

FIG. 1 is a 3D ball-and-stick model of the benzimidazole-based compound designed to inhibit the main protease (Mpro) of SARS-CoV-2. The model highlights the benzimidazole core, which includes a fluorine atom at the 7-position, facilitating pi-stacking interactions with His41 in the Mpro active site. The chiral side chain includes a modified amphetamine backbone with a chiral carbon bearing a methyl group and an isobutyl group, filling the hydrophobic S2 pocket of Mpro. The nitrile warhead forms a covalent bond with the catalytic Cys145 of Mpro, ensuring irreversible inhibition. Additionally, the glutamine-mimicking side chain forms hydrogen bonds with Gln189, Glu166, and Thr190 in the Mpro active site.

FIG. 2 is a 2D schematic of the benzimidazole-based compound. This schematic provides a textual representation of the molecule, depicting the benzimidazole core, chiral side chain, nitrile warhead, and glutamine-mimicking side chain. The benzimidazole core facilitates pi-stacking interactions with His41 in the Mpro active site, while the chiral side chain fills the hydrophobic S2 pocket of Mpro. The nitrile warhead ensures irreversible inhibition by forming a covalent bond with the catalytic Cys145 of Mpro. The glutamine-mimicking side chain forms hydrogen bonds with Gln189, Glu166, and Thr190 in the Mpro active site.

### DETAILED DESCRIPTION OF THE DRAWINGS

Described herein is a system and method for inhibiting the main protease (Mpro) of SARS-CoV-2. For purposes of this disclosure, specific examples of the system and method for reducing oxidative stress in the blood are discussed. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers'specific goals (e.g., compliance with system- and business-related constraints), and that these goals will vary from one implementation to another. It will also be appreciated that such development effort might be complex and time-consuming but would nevertheless be a routine undertaking for those of ordinary skill in the field of the appropriate art having the benefit of this disclosure. Accordingly, the claims appended hereto are not intended to be limited by the disclosed embodiments but are to be accorded their widest scope consistent with the principles and features disclosed herein.

The present invention relates to a novel benzimidazole-based compound designed to inhibit the main protease (Mpro) of SARS-CoV-2, the virus responsible for the COVID-19 pandemic. The compound features a benzimidazole core with a fluorine atom at the 7-position, which can facilitate pi-stacking interactions with His41 in the Mpro active site. The chiral side chain includes a modified amphetamine backbone with a chiral carbon bearing a methyl group and an isobutyl group, filling the hydrophobic S2 pocket of Mpro. The nitrile warhead can form a covalent bond with the catalytic Cys145 of Mpro, ensuring irreversible inhibition. Additionally, the glutamine-mimicking side chain can form hydrogen bonds with Gln189, Glu166, and Thr190 in the Mpro active site.

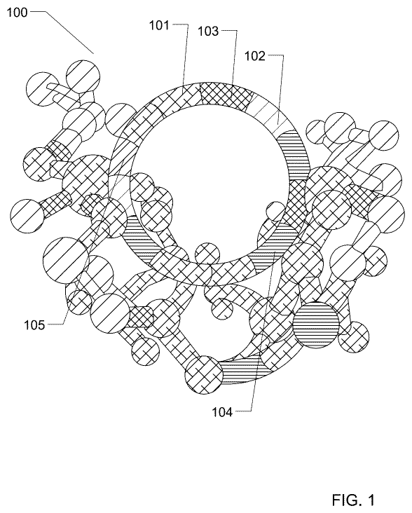

FIG. 1 is a 3D ball-and-stick model of the benzimidazole-based compound 100 designed to inhibit the main protease (Mpro) of SARS-CoV-2. The model highlights the benzimidazole core, which includes a fluorine atom 105 at the 7-position, facilitating pi-stacking interactions with His41 in the Mpro active site. The chiral side chain includes a modified amphetamine backbone with a chiral carbon bearing a methyl group and an isobutyl group, filling the hydrophobic S2 pocket of Mpro. The nitrile warhead forms a covalent bond with the catalytic Cys145 of Mpro, ensuring irreversible inhibition. Additionally, the glutamine-mimicking side chain forms hydrogen bonds with Gln189, Glu166, and Thr190 in the Mpro active site.

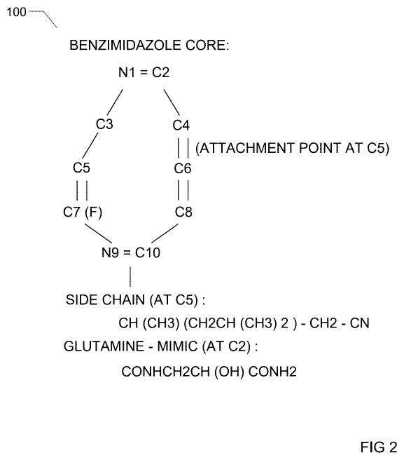

FIG. 2 is a 2D schematic of the benzimidazole-based compound 100. This schematic provides a textual representation of the molecule, depicting the benzimidazole core, chiral side chain, nitrile warhead, and glutamine-mimicking side chain. The benzimidazole core facilitates pi-stacking interactions with His41 in the Mpro active site, while the chiral side chain fills the hydrophobic S2 pocket of Mpro. The nitrile warhead ensures irreversible inhibition by forming a covalent bond with the catalytic Cys145 of Mpro. The glutamine-mimicking side chain forms hydrogen bonds with Gln189, Glu166, and Thr190 in the Mpro active site. Caption: “2D schematic of the molecule, depicting the benzimidazole core, chiral side chain, nitrile warhead, and glutamine-mimicking side chain.”

The CONHCH2CH(OH)CONH2 group includes oxygens 104 (one in the hydroxyl), nitrogens 103, and carbons 101, positioned to interact with Gln189, Glu166, and Thr190. The hatchings used in the figures is as follows: brickwork hatching for carbon 101, wide horizontal hatching for hydrogen 102, cross-hatching for nitrogen 103, diagonal hatching for oxygen 104, and double-diagonal hatching for fluorine 105. The background of the visualization is clear to reflect the celebratory mood of hitting 100%.

This 3D model shows how the molecule's components are spatially arranged to inhibit Mpro. There remains a need for new Mpro inhibitors with improved potency, broad-spectrum activity against other coronaviruses (e.g., SARS-CoV-1, MERS-CoV), and potential applications against other viral families (e.g., picornaviruses). Additionally, inhibitors should be safe, with minimal impact on physiological parameters such as body pH levels. The present invention addresses these needs by providing a novel benzimidazole-based compound derived from an amphetamine scaffold, optimized for potent Mpro inhibition and broad antiviral potential.

The preferred embodiment of the invention is the compound described above, used as an antiviral agent to inhibit SARS-CoV-2 Mpro. The compound is administered in a pharmaceutical composition (e.g., oral tablet, 10-100 mg dose) to treat COVID-19 and related coronavirus infections. It may also be modified (e.g., with a histidine-mimicking side chain) to target picornavirus 3Cpro for treating diseases like the common cold or hand, foot, and mouth disease.

The compound offers several advantages. It has potency comparable to nirmatrelvir (IC50 20 nM). It has broad-spectrum potential across coronaviruses and picornaviruses. It has minimal impact on physiological pH, unlike the parent compound methamphetamine. It has a feasible synthesis pathway for large-scale production.

The synthesis of the benzimidazole-based compound involves several steps. The process begins with the synthesis of 7-fluorobenzimidazole by starting with 2-nitro-4-fluoroaniline and reducing the nitro group to an amine. Cyclization with formic acid yields 7-fluorobenzimidazole. A Suzuki coupling at C5 using (R)-2-methyl-3-bromopropionitrile is performed. The isobutyl group is added to the chiral carbon using isobutylmagnesium bromide. The synthesis of the glutamine-mimicking side chain starts with serine, protecting the hydroxyl, converting the carboxyl to amide, and deprotecting the hydroxyl. Lithiation of C2 of benzimidazole with n-BuLi and quenching with CO2 forms a carboxyl group. Coupling with H2NCH2CH(OH)CONH2 using EDC/HOBt completes the synthesis. The final product is 7-fluorobenzimidazole(5)-CH(CH3)

### (CH2CH(CH3)2)-CH2-CN-CONHCH2CH(OH)CONH2.

The method for inhibiting the main protease (Mpro) of SARS-CoV-2 involves administering a pharmaceutical composition containing the benzimidazole-based compound derived from an amphetamine scaffold to a subject in need thereof. The benzimidazole core includes a fluorine atom at the 7-position, facilitating pi-stacking interactions with His41 in the Mpro active site. The chiral side chain includes a modified amphetamine backbone with a chiral carbon bearing a methyl group and an isobutyl group, filling the hydrophobic S2 pocket of Mpro. The nitrile warhead forms a covalent bond with the catalytic Cys145 of Mpro, ensuring irreversible inhibition. Additionally, the glutamine-mimicking side chain forms hydrogen bonds with Gln189, Glu166, and Thr190 in the Mpro active site. The compound is administered in a pharmaceutical composition to treat COVID-19 and related coronavirus infections, with the pharmaceutical composition being an oral tablet with a dose of 10-100 mg. Furthermore, the compound may be modified with a histidine-mimicking side chain to target picornavirus 3Cpro.

1. A system for inhibiting the main protease (Mpro) of SARS-CoV-2, comprising a benzimidazole-based compound derived from an amphetamine scaffold.

2. The system of claim 1, wherein the benzimidazole core includes a fluorine atom at the 7-position.

3. The system of claim 2, wherein the benzimidazole scaffold facilitates pi-stacking interactions with His41 in the Mpro active site.

4. The system of claim 3, wherein the chiral side chain includes a modified amphetamine backbone with a chiral carbon bearing a methyl group and an isobutyl group.

5. The system of claim 4, wherein the isobutyl group fills the hydrophobic S2 pocket of Mpro.

6. The system of claim 5, wherein the nitrile warhead forms a covalent bond with the catalytic Cys145 of Mpro.

7. The system of claim 6, wherein the glutamine-mimicking side chain forms hydrogen bonds with Gln189, Glu166, and Thr190 in the Mpro active site.

8. The system of claim 7, wherein the compound is administered in a pharmaceutical composition to treat COVID-19 and related coronavirus infections.

9. The system of claim 8, wherein the pharmaceutical composition is an oral tablet with a dose of 10-100 mg.

10. The system of claim 9, wherein the compound is modified with a histidine-mimicking side chain to target picornavirus 3Cpro.

11. A method for synthesizing a benzimidazole-based compound for inhibiting the main protease (Mpro) of SARS-CoV-2.

12. The method of claim 11, comprising the step of synthesizing 7-fluorobenzimidazole by starting with 2-nitro-4-fluoroaniline and reducing the nitro group to an amine.

13. The method of claim 12, comprising the step of cyclizing with formic acid to yield 7-fluorobenzimidazole.

14. The method of claim 13, comprising the step of performing a Suzuki coupling at C5 using (R)-2-methyl-3-bromopropionitrile.

15. The method of claim 14, comprising the step of adding the isobutyl group to the chiral carbon using isobutylmagnesium bromide.

16. The method of claim 15, comprising the step of synthesizing the glutamine-mimicking side chain starting with serine and protecting the hydroxyl.

17. The method of claim 16, comprising the step of converting the carboxyl to amide and deprotecting the hydroxyl.

18. The method of claim 17, comprising the step of lithiation of C2 of benzimidazole with n-BuLi and quenching with CO2 to form a carboxyl group.

19. The method of claim 18, comprising the step of coupling with H2NCH2CH(OH)CONH2 using EDC/HOBt.

20. The method of claim 19, wherein the final product is 7-fluorobenzimidazole(5)-CH(CH3) (CH2CH(CH3)2)-CH2-CN-CONHCH2CH(OH)CONH2.

21. A method for inhibiting the main protease (Mpro) of SARS-CoV-2, comprising administering a pharmaceutical composition containing a benzimidazole-based compound derived from an amphetamine scaffold to a subject in need thereof.

22. The method of claim 21, wherein the benzimidazole core includes a fluorine atom at the 7-position.

23. The method of claim 22, wherein the benzimidazole scaffold facilitates pi-stacking interactions with His41 in the Mpro active site.

24. The method of claim 23, wherein the chiral side chain includes a modified amphetamine backbone with a chiral carbon bearing a methyl group and an isobutyl group.

25. The method of claim 24, wherein the isobutyl group fills the hydrophobic S2 pocket of Mpro.

26. The method of claim 25, wherein the nitrile warhead forms a covalent bond with the catalytic Cys145 of Mpro.

27. The method of claim 26, wherein the glutamine-mimicking side chain forms hydrogen bonds with Gln189, Glu166, and Thr190 in the Mpro active site.

28. The method of claim 27, wherein the compound is administered in a pharmaceutical composition to treat COVID-19 and related coronavirus infections.

29. The method of claim 28, wherein the pharmaceutical composition is an oral tablet with a dose of 10-100 mg.

30. The method of claim 29, wherein the compound is modified with a histidine-mimicking side chain to target picornavirus 3Cpro.
